(19) United States
(12) Patent Application Publication
(10) Pub. No.: US 2004/0077885 A1
(43) Pub. Date: Apr. 22, 2004

(54) Catalyst regeneration by solvent extraction

(76) Inventors: Daniel Campos, Lancaster, PA (US); Richard Edward Ernst, Kennett Square, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/274,580

(22) Filed: Oct. 21, 2002

(51) Int. Cl. C07D 307/02; C07C 31/18

(52) U.S. Cl. 549/295; 549/429; 568/861

(57) ABSTRACT

A process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst, wherein a deactivated noble metal catalyst is regenerated by contacting the catalyst with a solvent, separating the solvent from the catalyst, and then reusing the regenerated catalyst in the process.

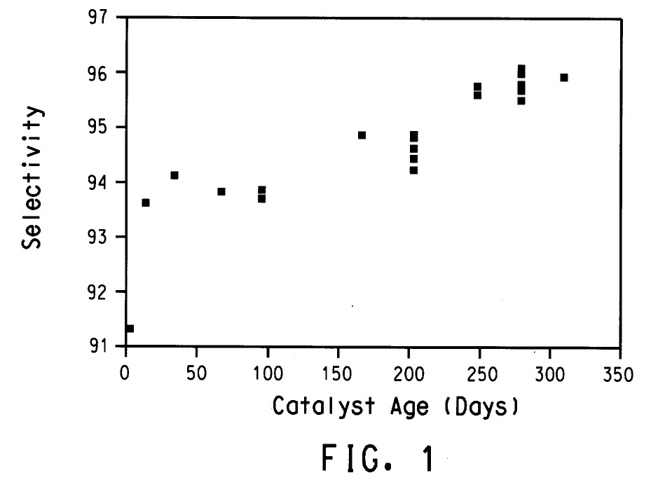

### BACKGROUND OF THE INVENTION

1. Field of the Invention

This invention relates to a process for the production of tetrahydrofuran, gamma butyrolactone, 1,4-butanediol and the like from a hydrogenatable precursor.

2. Description of the Related Art

Various methods and reaction systems have been proposed in the past for manufacturing tetrahydrofuran (THF) and 1,4 butanediol (BDO) by catalytic hydrogenation of gamma butyrolactone, maleic acid, maleic anhydride, succinic acid or related hydrogenatable precursors. Also, a variety of hydrogenation catalysts have been historically proposed for this purpose including various transition metals and their combinations deposited on various inert supports, all as generally known in the art. Many of these catalysts are proposed for use in hydrogenations carried out in an organic solvent or organic reaction media and not in an aqueous solution phase. In fact, at least one prior publication suggests that water and succinic acid may be considered as inhibitors to the desired catalysis; see Bulletin of Japan Petroleum Institute, Volume 12, pages 89 to 96 (1970).

A laid-open Japanese patent application (Kokai) 5-246915 directed to the aqueous phase catalytic hydrogenation of an organic carboxylic acid or ester teaches the use of any Group VIII noble metal, optionally in combination with either tin, rhenium or germanium, on a defined activated carbon support.

U.S. Pat. No. 5,698,749 discloses a process for producing 1,4-butanediol by aqueous hydrogenation of a hydrogenatable precursor using a catalyst comprised of a noble metal of Group VIII and at least one of rhenium, tungsten and molybdenum, on a carbon support pretreated with an oxidizing agent. The purpose of this pretreatment is to increase the yield of butanediol relative to gamma butyrolactone or tetrahydrofuran as compared to the use of a catalyst made with non-pretreated carbon. The reaction times stated in this patent are a very slow 9.5 hours.

On extended use of the aforementioned ruthenium-rhenium catalysts, the reaction rate for the hydrogenation decreases to the point where the deactivated or spent catalyst must be replaced with fresh catalyst. The old catalyst may then be destroyed by burning off the carbon, followed by partial recovery of the expensive metal ingredients. The overall cost of catalyst replacement is quite high. Similar deactivation problems are typically found with other noble metal catalysts in this process. There is either a need for a more economical method for recovery or for regeneration of such spent noble metal catalysts, or for extending the active life of such catalysts.

### SUMMARY OF THE INVENTION

This invention relates to an improved process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst. The improvement comprises the regeneration of deactivated noble metal catalyst by contacting the catalyst with a solvent, separating the solvent from the catalyst, and then reusing the regenerated catalyst in the stated process.

### BRIEF DESCRIPTION OF THE DRAWINGS

The FIGURE is a graph showing the effect of on-stream time on the selectivity of catalyst B.

### DETAILED DESCRIPTION OF THE INVENTION

In the production of tetrahydrofuran (THF), gamma butyrolactone (GBL) and 1,4 butanediol (BDO) by aqueous hydrogenation of maleic acid (MAC) and succinic acid (SAC), various noble metal catalysts are employed. For example, U.S. Pat. Nos. 5,478,952 and 6,008,384 disclose the use of specific ruthenium-rhenium catalysts in this process and both are incorporated by reference herein. For convenience, we will hereinafter refer to the ruthenium-rhenium catalyst of U.S. Pat. No. 5,478,952 as Catalyst-A (Cat-A) and the ruthenium-rhenium-tin catalyst of U.S. Pat. No. 6,008,384 as Catalyst-B (Cat-B). As is typical for noble metal hydrogenation catalysts, the activity and selectivity of these catalysts decline steadily with their use in production. They can lose a substantial part of their initial activity in a period as short as several months.

We have also found that one of the causes for this decrease in activity, as well as a decrease in selectivity, is the presence of carbon monoxide (CO), a known poison of transition-metal catalysts. It is believed that the CO is formed by decarbonylation and decarboxylation of reactants and intermediates during the hydrogenation reaction. Since excess hydrogen is recycled, the CO concentration in the recycled hydrogen tends to build up until it reaches a level in equilibrium with the losses from the hydrogen purge stream. The CO level may rise to 3000 parts per million (ppm) depending on time and the acid level in the reactor. Laboratory tests show that a CO level between 2000 and 3000 ppm depresses Cat-A catalyst activity quite significantly. This effect is somewhat smaller, but still significant for a Cat-B catalyst. A fresh catalyst may protect itself from CO poisoning by efficiently converting CO to innocuous CH₄ (methanation). However, within the first month of operation, a catalyst can lose more than half of its ability to convert CO to CH₄.

In the inventive process, a deactivated noble metal catalyst is contacted with a solvent to restore a major part of its previous activity. This solvent may be referred to as extraction solvent. The extraction solvent may be any liquid solvent that solubilizes the organic impurities that have built up in the catalyst. Preferably, the extraction solvent has a boiling point below 190° C. (at atmospheric pressure) when used in the continuous slurry regeneration system. Preferred solvents include alcohols, ketones, aldehydes, organic acids, ethers, esters, glycols, hydrocarbons, aromatics, furanes and water. THF is particularly preferred because of its solubilizing characteristics and because it is already present in the system. The contacting may be carried out with the catalyst supplied in either dried or slurry form and may be carried out within the reaction vessel or in a separate system. It can be done at any convenient temperature, however, for convenience, ambient or reflux temperatures are preferred. The spent solvent is then separated from the catalyst. The spent solvent may be removed by filtration, decantation or other means. It is preferred that, in the case of a slurry catalyst, the slurry to be treated contains about 20 to 50% solids for ease of handling.

In the case of a slurry reactor, a procedure can involve the following steps:

(1) separating the spent catalyst from the greater part of the reactor solvent.

This may be done in a number of ways. If it is done by decantation or distillation from the reactor itself, sufficient reactor solvent should be removed to reduce the reactor mass to a convenient level and make room for the addition of the extraction solvent. We have found it convenient to remove about half the volume in the reactor.

If it is done by filtration, any commercial filter medium that retains the catalyst should be adequate. Optionally, some extraction solvent may be added to the reactor slurry before the filtration. When using THF as extraction solvent, we have found that the addition of about an equal portion of THF to the reactor slurry makes the filtration faster and makes the resulting filter cake easier to handle. Other methods of separation, such as centrifugation, will be apparent to those skilled in the art. The reaction solvent removed during this separation step may be returned to the reactor when convenient.

(2) mixing, in a vessel equipped with agitation, the spent catalyst with an amount of extraction solvent sufficient to make the catalyst stirrable, and stirring with N₂ or H₂ recycle until equilibrium is reached.

We have found that in about 2 hours at ambient temperature the extraction step is essentially complete. If suitable, the reactor may be used for this step. Any convenient temperature may be used.

(3) stopping the stirring and letting the slurry settle until essentially all the catalyst has settled to a thick slurry.

We have found that 12 hours is an effective time for this step. More or less time may be required depending on the particle size and settling characteristics of the catalyst and the viscosity of the fluid.

(4) decanting and saving the essentially clear top layer of the extraction solvent.

(5) repeating steps (2) to (4) as necessary to obtain adequate catalyst regeneration.

We have found it optimum to repeat this step twice, that is, perform the step a total of three times. Either more times or fewer times may be required depending on the condition of the catalyst to be regenerated.

Preferably, the catalyst slurry from step (5) is returned to the reactor as slurry. Alternatively, it may be filtered or centrifuged to a cake and then returned to the reactor. If it is to be isolated as a cake, the extracted catalyst may be washed with water in the filter or centrifuge to remove the solvent for safety reasons before handling. However, some Re may be lost during the water wash.

When convenient, the extraction solvent from the decanted liquid from steps (4) and (5) may be recovered and purified. The decanted liquid contains the extraction solvent; the impurities removed from the catalyst; and possibly some small amount of catalyst metal. Depending on the solvent used, the extraction solvent may be purified for reuse by distillation; treatment with an adsorbent; by some other treatment; or it may be otherwise disposed of. Optionally, it may be filtered to remove any solid catalyst before the purification. We have found it convenient to purify the extraction solvent for reuse by distillation, but other methods may alternatively be used, as will be apparent to those skilled in the art. Optionally, any materials removed from the spent catalyst or solvent during the regeneration or purification steps may be further processed for recovery of any precious metals.

THF extraction can be done either in-situ or ex-situ on the whole catalyst charge between shutdowns. Also, it can be done periodically on portions of the charge removed from the reactor, with the portions returned to the reactor after THF extraction.

Alternatively, in a continuous slurry reactor system, a continuous procedure to regenerate a slurry catalyst in-situ with THF may be more convenient. It is to be recognized that many variations in the specific regeneration system design and regeneration procedure used will be apparent to one skilled in the art, and all such procedures are included within the scope of the present invention.

A preferred continuous regeneration procedure in such a system is described as follows:

(1) replacing the organic reactant feed with water while continuing hydrogen feed to the reactor and lowering the reactor temperature to 70° C. or lower.

Preferably, this step is continued until reactor acidity is zero.

(2) directing a flow of reactor slurry to a filter thickener to remove a filtrate portion and returning the remaining slurry back to the reactor along with newly added extraction solvent.

The slurry flowrate should be adequate to maintain flow of the solids in the filter thickener and prevent filter plugging. The extraction solvent added to the recycle loop should be in an amount equivalent to the filtrate amount so that the solids concentration of the reactor remains nearly constant.

(3) continuing step (2) for an effective period of time until the catalyst is regenerated.

Periodically, samples of reactor liquid should be taken to monitor the extraction solvent concentration. Based on laboratory tests with THF, a THF concentration in the liquid of about 95% is required to begin the effective extraction of impurities from the catalyst. After reaching an effective concentration in the reactor liquid, the process should be continued for several more hours to insure that the impurities are removed from the reactor liquid.

(4) discontinuing step (3), and gradually raising the reactor temperature while feeding water to replace evaporated extraction solvent.

This step should be continued until most of the extractive solvent in the reactor is displaced by water and the reactor temperature is around 190° C.

(5) resuming feed of organic reactants to the reactor.

When convenient, the spent extractive solvent may be sent to a refining operation to recover the solvent.

In the case of a fixed bed catalyst, the regeneration in-situ may be done by a process similar to that described above, except that the filtration loop is not needed. In this case, the extraction solvent would be fed to the reactor until the exiting liquid reaches the effective concentration for the extraction solvent, about 95% in the case of THF.

A preferred continuous regeneration procedure in such a system is described in the section below:

(1) replacing the organic reactant feed with water and lowering the reactor temperature to 70° C. or less while continuing hydrogen feed to the reactor.

(2) feeding extraction solvent to the fixed bed reactor and removing spent extraction solvent for an effective period of time until the catalyst is regenerated.

(3) discontinuing step (2) and gradually raising the reactor temperature while feeding water to replace evaporated extraction solvent.

(4) resuming feed of organic reactants to the reactor.

Surprisingly, the regenerated catalyst not only has nearly the same activity as new catalyst, but has substantially higher selectivity. The figure shows that new Cat-B catalyst selectivity is around 91.5%, but increases with age during a “break-in” period. The regenerated catalyst apparently does not need this “break-in” period. The higher initial selectivity of the regenerated catalyst, as shown in the examples below, is an additional advantage of the regeneration process.

The regeneration process is applicable to any noble metal catalyst used for the production of tetrahydrofuran, gamma butyrolactone, and 1,4 butanediol by aqueous hydrogenation of maleic acid and succinic acid. These noble metal catalysts include those containing, for example, ruthenium, rhenium, platinum, and palladium. Other metals such as tin may be present to aid in or modify the reaction. The metals will typically be on a support of carbon, alumina, silica or other support materials known in the art.

### EXAMPLES

The following examples were carried out on catalyst samples removed from a continuous reactor producing THF by the processes previously described.

### Examples 1-10

Various slurry samples containing 249-day old Cat-B catalyst (with nominally 2% Ru, 6% Re and 0.9% Sn) taken during operation from a continuous reactor are listed in Table 1. The extraction procedure consisted of mixing the slurry with an equal weight of THF followed by decantation. This was performed three times. The catalyst was then dried for accurate determination of catalyst amounts.

Each sample (0.4 g on dry basis) was tested in a 300-cc batch hydrogenation reactor at various CO levels. The tests were done with a feed mixture of H₂ and CO on 125 g of 7% SAC solution at 250° C. and 2000 psi, stirred at 700 RPM for 45 min. The pressure was held constant by continuous feed of the H₂/CO mixture throughout the test. After 45 min, the reactor was immediately cooled. Liquid and gas samples were analyzed by gas chromatograph.

The STY for a given species is determined as the difference between the final and initial moles of the species per unit time per unit mass of catalyst on a dry basis.

Comparative Examples A-H were not treated with any solvent. Examples 1-10 were extracted with THF and the slurry was extracted at ambient temperature.

TABLE 1

| | Feed ppm | Final ppm | CH4 STY | SAC STY | |
|---|---|---|---|---|---|
| Example | CO in H2 | CO in H2 | mol/hr-Kg | mol/hr-Kg | Selectivity % |
| A | 0 | 140 | 0.119 | 24.24 | 95.6 |
| B | 300 | 280 | 0.136 | 22.58 | 96.0 |
| C | 770 | 560 | 0.188 | 19.31 | 97.0 |
| D | 1100 | 810 | 0.204 | 18.90 | 95.5 |
| E | 1700 | 1350 | 0.153 | 15.84 | 97.1 |
| F | 2500 | 2000 | 0.222 | 11.88 | 96.7 |
| G | 3700 | 3000 | 0.204 | 11.50 | 95.8 |
| H | 5300 | 4500 | 0.221 | 9.52 | 94.6 |
| 1 | 0 | 60 | 0.561 | 33.08 | 95.0 |
| 2 | 0 | 70 | 0.527 | 33.21 | 95.0 |
| 3 | 0 | 60 | 0.612 | 34.67 | 94.9 |
| 4 | 300 | 130 | 0.747 | 32.03 | 95.3 |
| 5 | 770 | 260 | 1.019 | 31.14 | 96.4 |
| 6 | 1100 | 370 | 1.269 | 28.71 | 96.1 |
| 7 | 1700 | 700 | 1.209 | 23.47 | 97.1 |
| 8 | 2500 | 1100 | 1.701 | 20.39 | 97.0 |
| 9 | 3700 | 1800 | 1.807 | 16.59 | 97.0 |
| 10 | 5300 | 3000 | 1.880 | 13.27 | 96.8 |

The data above show that THF extraction significantly increases the SAC hydrogenation and methanation activities of the used catalyst. It is noted that at less than 500 to 1000 ppm CO it is difficult to discern the effect of THF extraction on selectivity. However, at higher than 1000 ppm CO, the THF-extracted Examples clearly show a much higher selectivity than for the non-extracted Comparative Examples.

### Examples 11-18

A spent catalyst, consisting of Cat-A catalyst with nominally 1% Ru, 6% Re on carbon only lasted 131 days and showed significant deactivation in the continuous reactor at the end of its life.

A portion of the spent catalyst slurry was THF extracted. This consisted of mixing the slurry with an equal weight of THF followed by decantation. This was performed three times. The catalyst was then dried for accurate weight determination. These are Examples 11-18. The activity loss was verified in the laboratory batch hydrogenation test with samples that were tested as-received (i.e., with no treatment whatever) and are designated as Comparative Examples I-Q.

The catalysts (“as is” and THF extracted) were tested in the batch hydrogenation reactor as follows: 0.4 g catalyst samples (on dry basis) were run with 7% SAC solution at 250° C. and 2000 Psi for 45 min at various CO levels. The results are presented in Table 2.

TABLE 2

| | Feed ppm | Final ppm | CH4 STY | -SAC STY | Selectivity |
|---|---|---|---|---|---|
| Example | CO in H2 | CO in H2 | mol/hr-Kg | mol/hr-Kg | % |
| I | 8100 | | 0.304 | 1.21 | 43.6 |
| J | 4800 | 4500 | 0.270 | 2.24 | 74.4 |
| K | 3300 | 2800 | 0.404 | 1.94 | 78.0 |
| L | 2100 | 1700 | 0.451 | 3.34 | 85.0 |
| M | 1500 | 1100 | 0.503 | 3.36 | 80.5 |
| N | 1020 | 610 | 0.488 | 5.01 | 83.5 |
| O | 550 | 280 | 0.417 | 5.88 | 85.0 |
| P | 330 | 160 | 0.284 | 5.66 | 83.8 |
| Q | 0 | 30 | 0.067 | 7.50 | 87.3 |
| 11 | 8000 | 5400 | 2.690 | 4.51 | 97.1 |
| 12 | 5000 | 1400 | 4.372 | 8.51 | 95.9 |
| 13 | 3300 | 680 | 4.191 | 12.48 | 95.8 |
| 14 | 2500 | 430 | 2.864 | 12.18 | 96.1 |
| 15 | 1600 | 350 | 2.010 | 12.21 | 96.2 |
| 16 | 1100 | 210 | 1.467 | 13.81 | 96.1 |
| 17 | 450 | 80 | 0.790 | 15.16 | 95.4 |
| 18 | 0 | 10 | 0.286 | 13.78 | 95.4 |

It is noted that at a CO level of zero, the THF-extracted catalyst activity approximates the activity of fresh Cat-A catalyst. This suggests that THF extraction restores most of the initial activity of a Cat-A deactivated catalyst while also increasing the selectivity and methanation activity substantially.

### Example 19-27

Various slurry samples containing used Cat-A catalyst (with nominally 1% Ru, 6% Re) taken during operation and after shutdown from a continuous reactor are listed in Table 3. Prior to activity measurement, Comparative Examples R-Y were prepared by simply drying the slurry overnight in a vacuum oven with N₂ purge. This treatment only removed water and volatile components, such as THF. Any foulants, such as waxes and other high boiling compounds remained on the catalyst.

Examples 19-27 were prepared wherein the slurry was extracted with THF under reflux and then filtered and dried. First the slurry sample, (normally 50 g of slurry) of about 20% solids was filtered in a Millipore funnel filter with 0.2 micrometer nylon membrane filter under vacuum (180 mm Hg) to about 50% solids. This is a slow process (overnight) as the filter cake is very sticky. Then the filter cake was transferred to a 500 ml round bottom flask and mixed with 5 parts of THF (based on 1 part by weight of original slurry). While stirring with a magnetic stirrer, the mixture was heated under reflux for 2 hours at about 66° C. The slurry was then filtered (same type of filter as above) and the resulting cake was dried at 110° C. in a vacuum oven for at least 6 hours.

All resulting samples were tested in the 300-cc batch hydrogenation reactor with pure H2 on a 7% SAC solution at 2000 psi, 250° C. and 700 RPM. The rate of conversion of SAC to GBL and by-products was measured and reported as STY in Table 3. In the case of the Comparative Examples, the STY was corrected for the real weight of catalyst, which excluded the weight of the organics remaining in the sample after drying. Also, the STY of the Comparative Examples was corrected by subtracting the STY obtained from runs using catalyst and water.

TABLE 3

| | Catalyst | | | |
|---|---|---|---|---|
| | age | Catalyst | STY (mol/hr- | |
| Example | (days) | description | Kg) | Selectivity |
| R | 4 | non-extracted | 21.3 | 93.2 |
| S | 63 | non-extracted | 13.1 | 94.1 |
| T | 63 | non-extracted | 14.8 | 93.0 |
| U | 63 | non-extracted | 13.7 | 92.9 |
| V | 76 | non-extracted | 11.4 | 93.3 |
| W | 80 | non-extracted | 12.0 | 93.1 |
| X | 104 | non-extracted | 8.8 | 94.7 |
| Y | 104 | non-extracted | 11.1 | 96.6 |
| 19 | 4 | THF extracted | 19.6 | 94.4 |
| 20 | 12 | THF extracted | 20.1 | 93.8 |
| 21 | 41 | THF extracted | 23.2 | 93.7 |
| 22 | 47 | THF extracted | 20.1 | 93.6 |
| 23 | 63 | THF extracted | 21.2 | 93.6 |
| 24 | 76 | THF extracted | 23.8 | 93.5 |
| 25 | 80 | THF extracted | 25.7 | 94.2 |
| 26 | 104 | THF extracted | 20.1 | 94.1 |
| 27 | 104 | THF extracted | 22.4 | 94.9 |

The THF-extracted examples had a STY of about 20 or greater throughout the catalyst's lifetime (which is the STY observed in fresh of Cat-A). On the other hand, the non-extracted Comparative Examples showed a steady decline so that at the end of the catalyst life, the STY decreased to between 9 and 11. This activity decline of about 50% would be compounded by the presence of CO in a continuous reactor (CO was not used in the batch tests).

### Example 28-32

Methylene chloride, acetone, methanol and toluene were compared to THF for reactivating samples of 249-day old Cat-B catalyst. The catalyst laboratory extraction procedure, which has been previously described, was the same for each solvent. A Comparative Example where no solvent was used is also included. The examples were tested as per the standard batch autoclave test, consisting of hydrogenating 125 g of 7% succinic acid (SAC) solution in the presence of 0.4 g of catalyst at 250° C. and 2000 psi and stirred at 700 RPM for 45 min. The pressure was held constant by continuous feed of H2. The STY for a given species is determined as the difference between the final and initial moles of said species per unit time per unit mass of catalyst. The results are presented in Table 4.

TABLE 4

| | | CH4 STY | | | |
|---|---|---|---|---|---|
| | | (mol/ | CO STY | SAC STY | Selectivity |
| Example | Solvent | hr-Kg) | (mol/hr-Kg) | (mol/hr-Kg) | % |
| Z | none | 0.137 | 0.257 | 26.79 | 95.8 |
| 28 | THF | 0.561 | 0.119 | 32.92 | 95.1 |
| 29 | CH3Cl | 0.513 | 0.103 | 33.23 | 95.7 |
| 30 | acetone | 0.513 | 0.120 | 31.13 | 95.7 |
| 31 | CH3OH | 0.424 | 0.085 | 32.07 | 95.0 |
| 32 | toluene | 0.546 | 0.154 | 33.44 | 95.8 |

The data show that methylene chloride, acetone, methanol and toluene (like THF) are also effective in reactivating Cat-B catalyst by solvent extraction.

We claim:

1. In a process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst, the improvement comprising the regeneration of deactivated noble metal catalyst by:
a) contacting the catalyst with a solvent,
b) separating the solvent from the catalyst, and
c) reusing the regenerated catalyst in the process.

2. In a process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst, the improvement comprising the regeneration of deactivated noble metal catalyst by:
(a) separating the deactivated catalyst from the greater part of the reactor solvent,
(b) mixing the catalyst with an amount of extraction solvent sufficient to make the catalyst stirrable in an agitated vessel,
(c) stirring with N₂ or recycled H₂ until equilibrium is reached,
(d) stopping the stirring to permit the slurry to settle until essentially all the catalyst has settled to a thick slurry,
(e) decanting the product of step (d),
(f) saving the essentially clear top layer of the extraction solvent, and
(g) repeating steps (b) to (f) as necessary to obtain adequate catalyst regeneration.

3. In a process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst, the improvement comprising the regeneration of deactivated noble metal catalyst carried out continuously in a slurry reactor system by:
(a) replacing the organic reactant feed with water and lowering the reactor temperature to 70° C. or lower while continuing hydrogen feed to a reactor,
(b) directing the reactor slurry to a filter thickener,
(c) removing a portion of the extraction solvent,
(d) returning the remainder of the slurry back to the reactor along with additional extraction solvent,
(e) repeating step (b) to (d) for an effective period of time until the catalyst is regenerated,
(f) discontinuing step (e), while gradually raising the reactor temperature while feeding water to replace evaporated extraction solvent, and
(g) resuming feed of organic reactants to the reactor.

4. In a process for production of tetrahydrofuran, gamma butyrolactone, 1,4 butanediol and the like from a hydrogenatable precursor such as maleic acid, succinic acid, corresponding esters and their mixtures and the like in an aqueous solution in the presence of hydrogen using a noble metal catalyst, the improvement comprising the regeneration of deactivated noble metal catalyst carried out continuously in a fixed bed reactor system by:
(a) replacing the organic reactant feed with water and lowering the reactor temperature to 70° C. or lower while continuing hydrogen feed to the reactor,
(b) feeding extraction solvent to the fixed bed reactor,
(c) removing spent extraction solvent for an effective period of time until the catalyst is regenerated,
(d) discontinuing steps (b) to (c) while gradually raising the reactor temperature while feeding water to replace evaporated extraction solvent, and
(e) resuming feed of organic reactants to the reactor.

5. The process of either of claim 1-4, wherein the solvent is selected from the group consisting of tetrahydrofuran, methylene chloride, acetone, methanol and toluene.

6. The process of claim 5, wherein the solvent is tetrahydrofuran.

7. The process of either of claims 1-4, wherein the catalyst contains at least one metal from the group consisting of ruthenium, rhenium, platinum, palladium and tin.

8. The process of claim 7, wherein the catalyst contains at least one noble metal.
